(19) United States
(12) Patent Application Publication
(10) Pub. No.: US 2004/0091875 A1
(43) Pub. Date: May 13, 2004

(54) Method for isolating nucleic acids

(76) Inventors: Martin Weber, Leichlingen (DE); Thorsten Singer, Solingen (DE); Sarah Cosaert, Koeln (DE)

Correspondence Address: Leon R Yankwich, Yankwich & Associates, 201 Broadway, Cambridge, MA, 02139, US

(21) Appl. No.: 10/332,667

(22) PCT Filed: Jul. 12, 2001
(86) PCT No.: PCT/EP01/08066

(30) Foreign Application Priority Data
Jul. 12, 2000 (DE) 100-33-991.3

(51) Int. Cl. C12Q 1/68

(52) U.S. Cl. 435/6

(57) ABSTRACT

The invention relates to a method for isolating nucleic acids from a solution, wherein the nucleic acids are adsorbed on a surface containing SiO2 in the presence of alkali halides and alcohol. The invention also relates to the use of a buffer solution containing alkali halides for isolating nucleic acids on a carrier containing SiO2, in addition to a kit for implementing a method for isolating nucleic acids from a solution.

The present invention relates to a process for isolating nucleic acids from a solution, in which the nucleic acids are adsorbed onto a surface containing SiO₂.

The present invention further relates to the use of a buffer solution for isolating nucleic acids on a carrier containing SiO₂ and a kit for carrying out a process for isolating nucleic acids from a solution.

The purification and isolation of nucleic acids on mineral carriers in the presence of chaotropic salts is well known in the literature.

Marko et al. [Analyt. Biochem. 121 (1982) 382] and Vogelstein et al. [Proc. Nat. Acad. Sci. 76 (1979) 615] recognised that if the DNA from extracts containing nucleic acid is exposed to high concentrations of sodium iodide or sodium perchlorate, only the DNA binds to mechanically finely divided glass scintillation tubes and comminuted glass fibre membranes or glass fibre plates, whereas RNA and proteins do not bind. The DNA thus bound can be eluted with water, if desired.

EP 0389063 B1 relates to a process for isolating nucleic acids from a biological source. According to this method the biological sources containing DNA, such as blood, cells, plasma, etc., are lysed in the presence of chaotropic salts in high concentrations and then the nucleic acids are bound to a silica surface. They are then washed and eluted.

The process described in U.S. Pat. No. 5,155,018 describes the isolation of RNA from biological sources which contain DNA and other ingredients in addition to RNA.

The biological sample is acidified and mixed with a chaotropic agent such as a guanidinium salt. Silicate particles are added to the sample and under the conditions specified RNA binds to the silicate particles. Then, again, the RNA is also separated from the particles.

In WO 95/01359 Colpan et al disclose a process for purifying and separating nucleic acid mixtures by adsorbing the nucleic acid from an alcohol-containing solution with a high ionic strength. The adsorption solution contains, in addition to alcohol in a concentration of 1 to 50 vol. %, salts in a concentration of 1 to 10 M, of which the chaotropic salts such as guanidinium thiocyanate, sodium perchiorate or guanidinium hydrochloride are preferred.

WO 95/21849 relates to a process for separating double- and/or single-stranded nucleic acids from sources which contain these nucleic acids. In this process, too, the nucleic acids are adsorbed on mineral carriers under conditions which allow binding of the desired type of nucleic acid, while the unwanted type of nucleic acid does not bind to this mineral carrier.

In order to bind predominantly single-stranded nucleic acid to a mineral carrier and thus separate it from double-stranded nucleic acid, the treatment conditions are adjusted with an aqueous mixture of salts, particularly chaotropic salts, and alcohol, in accordance with the samples containing the two types of nucleic acid. The double-stranded nucleic acid which is not adsorbed can then be further purified or isolated by known methods.

Nucleic acids which are used for molecular-biological applications, such as PCR, sequencing and gene transfer, particularly for transfection or vaccination, are subject to extremely stringent requirements with regard to purity and integrity. The use of nucleic acids in molecular diagnostics or molecular medicine presupposes that they are free from toxic substances which may lead, for example, to pathogenic effects in the organisms which are to be treated.

One factor which is common to the processes known from the prior art is that chaotropic salts are used in high concentrations for isolating nucleic acids on silica surfaces. Chaotropic substances such as guanidinium hydrochloride, guanidinium thiocyanate or sodium perchlorate are highly toxic substances.

The possibility that the nucleic acids isolated in the presence of these substances will be contaminated therewith and thus made unusable or largely unusable for use in molecular biological applications cannot be ruled out.

The handling of chaotropic substances also constitutes a major risk to the health of the user, which means that certain safety precautions have to be taken when handling these substances.

The technical problem on which the present invention is based is to provide an improved process which overcomes the disadvantages known from the prior art. The substances used in this process for binding the nucleic acid should not be toxic. At the same time the process should be as low-cost as possible, for example by using cheap chemicals, and the isolated nucleic acids should be isolated in a quantitatively and qualitatively pure form.

Surprisingly, the present invention provides a process for isolating nucleic acids from a solution, which solves this problem. The invention resides in the fact that in a first step the binding of nucleic acids to surfaces containing SiO₂ is carried out in the presence of alkali metal halides in a concentration of 0.1 to 3 M, preferably 0.25-1.5 M, and alcohol in a concentration of 37 to 70 vol. %. The nucleic acids adsorbed onto the surface containing SiO₂ are then optionally washed with an alcohol-containing washing buffer and the nucleic acid is eluted with an aqueous salt solution or with water.

To bind the nucleic acids onto the surface containing SiO₂, aqueous adsorption solutions are used which contain alkali metal halides such as NaCl, KCl and LiCl in a concentration of 0.1 to 3 M, preferably 0.25-1.5 M, more preferably 0.5-1.25 M and particularly 0.5-1.0 M. Alkali metal halides are non-toxic substances and the handling of the salt solutions in the concentration used is perfectly safe in terms of health.

The aqueous adsorption solutions contain, in addition to the abovementioned salts, lower aliphatic, branched or unbranched alcohols with a chain length of 1 to 5 carbon atoms. The aliphatic alcohols contained in the solution are preferably methanol, ethanol, propanol, isopropanol and butanol in a concentration of 37-70 vol. %, preferably 37-50 vol. %. Of the abovementioned alcohols, ethanol and/or isopropanol in a concentration of 37-70 vol. % are particularly preferred.

Surfaces containing SiO₂ may be for example porous or non-porous silicon oxides or metal-silicon mixed oxides, silica gels, materials based on glass, e.g. modified or unmodified glass particles or ground glass, quartz, zeolites or mixtures of one or more of the abovementioned substances.

By a surface is meant, for the purposes of the present invention, any microporous boundary layer.

In a particularly preferred embodiment of the process according to the invention, the surface containing SiO₂ is a porous membrane or a filter made of silica gel, glass fibres or quartz fibres.

In another embodiment of the process according to the invention, the term surface in the wider sense also includes a layer of particles or granules or fibres, such as e.g. silica gel fleece.

The bound nucleic acid may be eluted according to the invention using water or aqueous saline solutions as eluant. The saline solutions used are buffer solutions known from the prior art, such as, for example, morpholinopropanesulphonic acid (MOPS), tris(hydroxymethyl)aminomethane (TRIS), 2-[4-(2-hydroxyethyl)-1-piperazino]ethanesulphonic acid (HEPES) in a concentration of 0.001 to 0.5 mol/litre, preferably 0.01 to 0.2 mol/litre, most preferably 0.01 to 0.05 molar solutions.

In another embodiment of the process according to the invention, the nucleic acids contained in the eluate may preferably be isolated by alcoholic precipitation.

The nucleic acids isolated by this process are free from toxic substances and are thus suitable for use in molecular biology.

The term “nucleic acid” should hereinafter be understood in its widest sense, i.e. to include ribonucleic acids (RNA) and also deoxyribonucleic acids (DNA) in all lengths and configurations, such as double-stranded, single-stranded, circular and linear, branched, etc., and all possible subunits thereof, such as e.g. monomeric nucleotides, oligomers, plasmids, viral and bacterial DNA and RNA, as well as genomic and non-genomic DNA and RNA from animal and plant cells or other eukaryotes, mRNA in processed and unprocessed form, tRNA, hn-RNA, rRNA, cDNA as well as all other conceivable nucleic acids.

The process according to the invention makes it possible to isolate nucleic acids of every origin from solutions. The sample containing nucleic acids originates, for example, from animal or plant tissues, tissue or cell cultures, bone marrow, human and animal body fluids such as blood, serum, plasma, urine, sperm, cerebrospinal fluid, sputum and smears, plants, parts of plants and plant extracts, e.g. juices, fungi, procaryotic or eucaryotic microorganisms such as bacteria or yeasts, fossilised or mummified samples, soil samples, clarified sludge, waste water and foodstuffs (particularly processed, i.e. industrially prepared foodstuffs). Nucleic acids formed by chemical reactions, e.g. those obtained by polymerase chain reaction (PCR) or plasmid-DNA, genomic DNA and RNA and/or nucleic acids which originate from microorganisms may also be isolated according to the invention.

The process according to the invention is particularly suitable for isolating plasmid DNA from bacteria, such as e.g. E. coli for subsequent cloning, transfection or sequencing.

The lysing of the bacteria is effected using known lysing methods such as, for example, alkaline lysing according to Bimboim and Doly (1979) or lysing by heating according to Holmes and Quigley.

The cell debris as well as the precipitated proteins and the genomic DNA are eliminated from the viscous lysate by centrifuging or filtering and a clarified lysate is obtained which contains the plasmid DNA. The plasmid DNA can be purified by ion exchange chromatography, for example, and the plasmid DNA thus pre-purified can then be isolated using the process according to the invention.

The invention further relates to a kit for isolating nucleic acids from a solution, comprising

a) an adsorption solution containing 0.25-1.5 M NaCl, KCl or a mixture thereof and ethanol or isopropanol in a concentration of 37-70 vol. %, and

b) a surface containing SiO₂.

The surface containing SiO₂ may be a porous membrane or a filter made of silica gel, glass fibres or quartz fibres and may be arranged in a suitable apparatus. The kit preferably additionally contains solutions which are suitable for lysing, as well as washing and elution buffers as described above.

The nucleic acids isolated according to the invention are free from enzymes that break down nucleic acids and are therefore sufficiently pure that they can immediately be further treated and processed in various ways.

The nucleic acids produced according to the invention may be used for cloning and act as substrates for all kinds of enzymes, such as for example DNA polymerases, DNA restriction enzymes, DNA ligase and reverse transcriptase.

The nucleic acids prepared by the process according to the invention are particularly suitable for amplification, especially PCR, Strand Displacement Amplification, the Rolling Circle process, Ligase Chain Reaction (LCR) and similar processes.

The process according to the invention is also particularly suitable for preparing nucleic acids for use in diagnostics, particularly for a method of diagnosis which is characterised in that the nucleic acid purified by the process according to the invention is amplified in a subsequent step and then and/or at the same time the nucleic acid thus amplified is detected (e.g. Holland, P. M. et al., 1991, Proc. Natl. Acad. Sci. 88, 7276-7280. Livak, K. J. et al., 1995. PCR Methods Applic. 4, 357-362; Kievits, T. et al.., 1991. J. Virol. Meth. 35, 273-286; Uyttendaele, M. et al., 1994. J. Appl. Bacteriol. 77, 694-701).

### EXAMPLE 1

Isolation of Plasmid DNA in the Presence of NaCl and Alcohol in Various Concentrations

390 μl aliquots of the individual buffers (0.25-1.5 M NaCl; 50 mM Tris, pH 8.5; 15% (w/v) isopropanol) were mixed with 1-μg of plasmid DNA (pCMVβ; Messrs Clontech #6177-1) (c=1 μg/μl) and combined with various amounts of isopropanol, corresponding to a total amount of 28.9-49.8 vol % of isopropanol in the binding buffers in question. After five minutes' incubation at ambient temperature (20-25° C.) the mixtures were transferred into a column containing a silica membrane and passed through the silica membrane in vacuo (about 600 mbar; using the QIAvac 6S of Messrs QIAGEN GmbH). Then it was washed with 750 μl of PE buffer (10 mM Tris, pH 7.5; 80% ethanol) and air was passed through the membrane until it dried. Elution was carried out by the addition of 100 μl of EB buffer (10 mM Tris, pH 8.5) and the yield was determined photometrically at 260 nm. The results are shown in Table 1.

TABLE 1

| Isopropanol | | | | | | | |
|---|---|---|---|---|---|---|---|
| (total) | C_{NaCl} | 0.25 M | 0.5 M | 0.75 M | 1.0 M | 1.25 M | 1.5 M |
| 28.9 vol % | DNA | 4.7[μg] | 5.8[μg] | 8.0[μg] | 7.3[μg] | 7.6[μg] | — |
| | OD₂₆₀ | 0.0934 | 0.1154 | 0.1593 | 0.1456 | 0.1522 | |
| 34.3 vol % | DNA | 8.2[μg] | 8.1[μg] | 7.4[μg] | 7.5[μg] | 7.0[μg] | 6.4[μg] |
| | OD₂₆₀ | 0.1644 | 0.1611 | 0.1484 | 0.1491 | 0.1390 | 0.1493 |
| 39.0 vol % | DNA | 6.2 g[μg] | 5.8[μg] | 8.0[μg] | 6.2[μg] | 7.3[μg] | 5.8[μg] |
| | OD₂₆₀ | 0.1245 | 0.1161 | 0.1603 | 0.1244 | 0.1452 | 0.1211 |
| 43.1 vol % | DNA | 7.2[μg] | 7.7[μg] | 6.2[μg] | 6.9[μg] | 7.4[μg] | 5.5[μg] |
| | OD₂₆₀ | 0.1444 | 0.1534 | 0.1231 | 0.1375 | 0.1469 | 0.1100 |
| 46.6 vol % | DNA | 5.7[μg] | 8.4[μg] | 7.3[μg] | 8.5[μg] | 7.9[μg] | 5.6[μg] |
| | OD₂₆₀ | 0.1129 | 0.1669 | 0.1454 | 0.1701 | 0.1585 | 0.1104 |
| 49.8 vol % | DNA | 6.5[μg] | 6.0[μg] | 6.1[μg] | 6.3[μg] | 6.3[μg] | 3.3[μg] |
| | OD₂₆₀ | 0.1294 | 0.1200 | 0.1221 | 0.1252 | 0.1258 | 0.0719 |

Dilution factor 20; total volume 50 μl; DNA yield 10 μg = 100%

### EXAMPLE 2

Isolation of Plasmid DNA at Different Alcohol Concentrations

500 μg (c=1 μg/μl) of plasmid DNA (pCMVβ; Messrs Clontech #6177-1) were dissolved in 5 ml of Q1 buffer (1.25 M NaCl; 50 mM Tris, pH 8.5; 15 vol % isopropanol). Then isopropanol was added in an amount corresponding to a total content of isopropanol in the binding buffer of 22.7-43.3 vol %, carefully mixed and incubated for 5 min at room temperature (20-25° C.). The DNA/Q1/isopropanol mixture was pressed into a glass fibre filter (Messrs Sartorius, Minisart Series) and the membranes were blown dry. Elution was carried out with 1 ml of TE buffer (10 mM Tris, pH 7.5; 1 mM EDTA). The yield was determined photometrically at 260 nm. The results are shown in Table 2.

TABLE 2

| Isopropanol | | | |
|---|---|---|---|
| (total) | DNA [μg] | DNA [%] | OD₂₆₀ values |
| 22.7 vol % | 242 | 48 | 0.2419 |
| 29.2 vol % | 416 | 83 | 0.4159 |
| 34.6 vol % | 383 | 77 | 0.3826 |
| 39.3 vol % | 415 | 83 | 0.4146 |
| 43.3 vol % | 412 | 82 | 0.4118 |

dilution factor: 20; total volume: 1 ml

### EXAMPLE 3

Isolation of Plasmid DNA in the Presence of KCl and Alcohol in Various Concentrations

390 μl aliquots of the individual buffers (0.25-1.0 M KCl; 50 mM Tris, pH 8.5; 15% (w/v) isopropanol) were mixed with 10 μg of plasmid DNA (pCMVβ;

Messrs Clontech #6177-1) (c=1 μg/μl) and combined with various amounts of isopropanol, corresponding to an amount of up to 57.3 vol % of isopropanol (see Table 3) or up to 71.4 vol % of isopropanol together with ethanol in the binding buffers in question. After five minutes' incubation at ambient temperature (20-25° C.) the mixtures were transferred into a column containing a silica membrane (QIAquick made by Messrs QIAGEN GmbH, #28104) and passed through the silica membrane in vacuo (about 600 mbar; using QIAvac 6S of Messrs QIAGEN GmbH). Then it was washed with 750 μl of PE buffer (10 mM Tris, pH 7.5; 80% ethanol) and air was passed through the membrane until it dried. The mixture was eluted by the addition of 100 μl of EB buffer (10 mM Tris, pH 8.5) and the yield was determined photometrically at 260 nm.

TABLE 3

| | Isopropanol | DNA yield | | ethanol/ | DNA yield | |
|---|---|---|---|---|---|---|
| C_{KCl} | (total) | [μg] | OD₂₆₀ | isopropanol | [μg] | OD₂₆₀ |
| 0.25 M | 46.6 vol % | 9.5 | 0.1185 | 66.7/5.9 vol % | 9.7 | 0.1208 |
| 0.5 M | 49.8 vol % | 8.8 | 0.1101 | 71.4/4.2 vol % | 10.0 | 0.1355 |
| 0.75 M | 46.6 vol % | 8.8 | 0.1098 | 60.0/5.9 vol % | 10.0 | 0.1322 |
| 0.75 M | 57.3 vol % | 9.6 | 0.1194 | 66.7/4.2 vol % | 10.0 | 0.1770 |
| 1.0 M | 43.1 vol % | 10.0 | 0.1727 | 33.3/9.8 vol % | 10.0 | 0.1280 |

dilution factor 20; total volume 80 μl.

### EXAMPLE 4

Isolation of Plasmid DNA in the Presence of LiCl; NaCl or KCl and Alcohol in Various Concentrations

390 μl aliquots of the individual buffers (0.25-1.0 M salt; 50 mM Tris, pH 8.5; 15% (w/v) isopropanol) were mixed with 10 μl (≈10 μg) of plasmid DNA (pCMVβ; Messrs Clontech #6177-1) (c=1 μg/μl) and combined with various amounts of ethanol, corresponding to an amount of up to 71.4 vol % of ethanol (see Table 4 below) in the binding buffers in question. After five minutes' incubation at ambient temperature (20-25° C.) the mixtures were transferred into a column containing a silica membrane and passed through the silica membrane in vacuo (about 600 mbar; using QIAvac 6S of Messrs QIAGEN GmbH). Then it was washed with 750 μl of PE buffer (10 mM Tris, pH 7.5; 80% ethanol) and air was passed through the membrane until it dried. The mixture was eluted by the addition of 100 μl of EB buffer (10 mM Tris, pH 8.5) and the yield was determined photometrically at 260 nm.

TABLE 4

| | LiCl | | NaCl | | | |
|---|---|---|---|---|---|---|
| | DNA | | DNA | | KCl | |
| [Salt]/[EtOH] | [μg] | OD₂₆₀ | [μg] | OD₂₆₀ | [μg] | OD₂₆₀ |
| 0.25 M/66.7 vol % | 7.2 | 0.0721 | 7.6 | 0.0763 | 10.0 | 0.1087 |
| 0.5 M/71.4 vol % | 7.9 | 0.0794 | 7.8 | 0.0783 | 10.0 | 0.1005 |
| 0.75 M/60 vol % | 9.0 | 0.0895 | 8.6 | 0.0862 | 8.8 | 0.0875 |
| 0.75 M/66.7 vol % | 9.0 | 0.0857 | 7.7 | 0.0771 | 10.0 | 0.1001 |
| 1.0 M/33.3 vol % | 9.6 | 0.0963 | 10.0 | 0.1253 | 8.9 | 0.0890 |

dilution factor 20; total volume 100 μl.

### EXAMPLE 5

Isolation of Larger Amounts of DNA

Increasing amounts of plasmid DNA (c=1 μg/μl; pCMVβ; Messrs Clontech #6177-1) were dissolved in 15 ml of Q1 buffer (1.25 M NaCl; 50 mM Tris, pH 8.5; 15 vol % isopropanol). Then isopropanol was added to give a final concentration of 49.8 vol %, the ingredients were carefully mixed and incubated for 5 minutes on the laboratory bench.

The DNA/Q1/isopropanol mixture was transferred into a 20 ml syringe fitted with a syringe pre-filter containing a silica membrane (Messrs Sartorius, Minisart Series). The mixture was forced through the filter under uniform pressure, the filter was removed and air was blown through the membrane again to remove the alcohol residues. Elution was carried out by forcing 5 ml of TE buffer (10 mM Tris, pH 7.5; 1 mM EDTA) through the syringe pre-filter using a fresh 5 ml syringe. The yield was determined photometrically at 260 nm. The results are shown in Table 5.

TABLE 5

| | | | | |
|---|---|---|---|---|
| pCMVb [mg] | 1.5 | 2.0 | 2.5 | 3.0 |
| DNA yield | 1.274 | 1.577 | 2.367 | 2.523 |
| [mg] | | | | |
| DNA yield | 85 | 79 | 95 | 84 |
| [%] | | | | |
| OD₂₆₀ | 0.2547 | 0.3153 | 0.4733 | 0.5046 |

Dilution factor 20; total volume 5 ml

Results:

The recovery rate of the DNA is between 80 and 90% even with larger quantities of plasmid DNA.

### EXAMPLE 6

Isolation of Plasmid DNA in the Presence of Various Concentrations of NaCl and Alcohol

340 μl aliquots of an NaCl solution (0 M; 0.1 M; 0.25M; 0.5 M; 1M; 2.5 M; 5 M; saturated solution; 50 mM Tris, pH 8.5) were mixed with 10 μl of a solution containing 1 μg/μl plasmid DNA (pCMVβ; Messrs Clontech #6177-1). 350 μl of isopropanol were added to each sample, resulting in a concentration of 50 vol. % for each mixture. After five minutes' incubation at ambient temperature (20-25° C.) the mixture was transferred into a column (QIAvac 6S; QIAGEN GmbH) containing a glass fibre/silica membrane (QIAprep 8-well strips; QIAGEN GmbH). The DNA-containing solutions were passed through the membrane in vacuo (about 600 mbar) and then eluted with 200 μl of EB buffer (10 mM Tris, pH 8.5). The yields of DNA (as a percentage of the amount put in) in the quadruple measurements taken at each salt concentration as well as the average are shown in Table 6 below.

TABLE 6
Yields [%]:

| | value 1 | value | value | value | average | STDEV |
|---|---|---|---|---|---|---|
| 0 M | 17 | 16 | 19 | 18 | 17.5 | 1.291 |
| 0.1 M | 100 | 100 | 100 | 100 | 100 | 0.000 |
| 0.25 M | 100 | 100 | 100 | 100 | 100 | 0.000 |
| 0.5 M | 100 | 100 | 100 | 100 | 100 | 0.000 |
| 1 M | 100 | 100 | 100 | 100 | 100 | 0.000 |
| 2.5 M | 100 | 100 | 100 | 100 | 100 | 0.000 |
| 5 M | 70 | 73 | 74 | 73 | 72.5 | 1.732 |
| saturated | 83 | 68 | 75 | 79 | 76.3 | 6.397 |

STDEV: standard deviation, starting from a random sample

The result of this test shows that in the recovery of DNA the yield is reduced at very high salt concentrations (5 M, saturated) by comparison with lower salt concentrations of between 0.1 and 2.5 M.

### EXAMPLE 7

Isolation of Plasmid DNA in the Presence of Various Salts or Various Alcohol Concentrations

500 μg aliquots of plasmid DNA were dissolved in 5 ml of TE buffer (10 mM Tris-Cl; pH 8.0; 1 mM EDTA; QIAGEN GmbH) and isopropanol was added until the final concentrations of alcohol in the individual mixtures were 41.2, 50, 66.7 and 75 vol-%. All the measurements for the DNA isolation were carried out with a QIAprecipitator Maxi (QIAGEN GmbH) in a QIAvec 6S column (QIAGEN GmbH). Table 7 shows the results of the tests, in which the salt concentration in the different mixtures was 1.25 M NaCl. The tests were each carried out several times, and Table 7 shows the results for the individual samples and the average with standard deviation calculated therefrom.

TABLE 7
Yields [%]:

| | | | | |
|---|---|---|---|---|
| Vol % isopropanol | 41.2 | 50 | 66.7 | 75 |
| 1 | 81.1 | 100 | 60.6 | 37.3 |
| 2 | 93.1 | 93.2 | 69.9 | 42.9 |
| 3 | 67.7 | 77.2 | 86.4 | 75.5 |
| 4 | 55.2 | 70.5 | 88.4 | 67.1 |
| 5 | | 71.8 | 68 | 63 |
| 6 | | 51.4 | 65 | 60.6 |
| averages [%] | 74.3 | 77.4 | 73.1 | 57.7 |
| STDEV | 16.412 | 15.892 | 10.560 | 13.397 |

Then the experiments described above were modified by changing the nature of the salt solution used: the 1.25 M NaCl solution was replaced by a 1.25 M KCl solution. The results of the experiments with KCl solution are shown in table 8.

TABLE 8
Yields [%]:

| | | | | |
|---|---|---|---|---|
| Vol % isopropanol | 41.2 | 50 | 66.7 | 75 |
| 1 | 70.9 | 82.9 | 76.4 | 27 |
| 2 | 69 | 79.8 | 66.8 | 37 |
| 3 | 66.1 | 75.1 | 79.0 | 84.7 |
| 4 | 68.9 | 72.6 | 76.7 | 65.4 |
| 5 | | 68.8 | 63.8 | 44.8 |
| 6 | | 70.6 | 67.6 | 55.2 |
| averages [%] | 68.7 | 75.0 | 71.7 | 52.4 |
| STDEV | 1.977 | 4.984 | 5.817 | 18.975 |

Finally, the influence of the buffer on the yield of plasmid DNA was also investigated. The method described above was used, and in the mixture containing a 1.25 M NaCl solution the TE buffer was replaced by a QF buffer (50 mM MOPS; pH 17.0; 15% Isopropanol). The results of the multiple measurements together with the average and standard deviation are shown in Table 9. The difference in the alcohol concentrations in the individual mixtures from those in Tables 7 and 8 is due to the 15% isopropanol content of the QF buffer.

TABLE 9
Yields [%]:

| | | | | |
|---|---|---|---|---|
| Vol % isopropanol | 22.7 | 50 | 57.5 | 78.8 |
| 1 | 59.6 | 79.8 | 71.7 | 39.2 |
| 2 | 40.7 | 78 | 82.2 | 39 |
| 3 | 24.4 | 91.8 | 76.8 | 69.1 |
| 4 | 25.6 | 57 | 79.8 | 67.8 |
| 5 | | | 72.2 | 68.4 |
| 6 | | | 49.2 | 55.6 |
| averages [%] | 37.6 | 76.7 | 72.0 | 56.5 |
| STDEV | 16.450 | 14.461 | 10.862 | 13.130 |

The results show that the nature of the salt used (NaCl/KCl) has only a slight effect on the yield in the isolation of DNA. Similarly, replacement of the buffer has virtually no effect on the yield of DNA (77.4% for 1.25 M NaCl and TE buffer and 75.0% for 1.25 M NaCl and QF buffer).

1. Process for isolating nucleic acids from a solution, comprising the following steps:
a) adsorbing the nucleic acids contained in the solution on a surface containing SiO₂ in the presence of alkali metal and/or alkaline earth metal salts,
b) optionally washing the nucleic acids adsorbed onto the surface containing SiO₂ with an alcohol-containing washing buffer, and
c) eluting the nucleic acids with an aqueous solution and optionally isolating the nucleic acids,
characterised in that in step a) the adsorption of the nucleic acids onto the surface containing SiO₂ is carried out in the presence of 0.1 to 3 M alkali metal and/or alkaline earth metal salts and 37 to 70 vol % of an aliphatic alcohol;

2. Process according to claim 1, characterised in that the nucleic acid is plasmid DNA.

3. Process according to one of claims 1 or 2, characterised in that the surface containing SiO₂ consists of silica gel, glass fibres or quartz fibres.

4. Process according to one of claims 1 to 3, characterised in that the surface containing SiO₂ is a membrane or a filter.

5. Process according to one of claims 1 to 4, characterised in that the alkali metal salts contained in the solution are halides, preferably NaCl and/or KCl.

6. Process according to one of claims 1 to 5, characterised in that in step a) NaCl is present in the solution in a concentration of from 0.25 to 1.5 M, preferably from 0.5 to 1.25 M and most preferably from 0.5 to 1.0 M.

7. Process according to one of claims 1 to 6, characterised in that the alcohols contained in the solution are lower aliphatic, branched or unbranched alcohols with a chain length of 1 to 5 carbon atoms.

8. Process according to claim 7, characterised in that the alcohol contained in the solution is ethanol and/or isopropanol.

9. Process according to one of claims 1 to 8, characterised in that the eluant solutions used in step c) contain morpholinopropanesulphonic acid (MOPS), tris(hydroxymethyl)aminomethane (TRIS) or 2-[4-(2-hydroxyethyl)-1-piperazino]ethanesulphonic acid (HEPES) in a concentration of 0.001 to 0.5 mol/litre, preferably 0.001 to 0.2 mol/litre, most preferably 0.01 to 0.05 mol/litre.

10. Use of a solution containing 0.25-1.5 M NaCl, KCl or a mixture thereof and ethanol or isopropanol in a concentration of 37-70 vol. % for the adsorption of nucleic acid onto a surface containing SiO₂.

11. Kit for carrying out the process according to one of claims 1 to 9, containing
a) a solution according to claim 10 and
b) a surface containing SiO₂.
